(19) United States
(12) Patent Application Publication
(10) Pub. No.: US 2011/0166610 A1
(43) Pub. Date: Jul. 7, 2011

(54) SYSTEMS AND METHODS FOR STABILIZATION OF BONE STRUCTURES, INCLUDING THOROCOLUMBAR STABILIZATION SYSTEMS AND METHODS

(76) Inventors: Moti Altarac, Irvine, CA (US); Joey Camia Reglos, Lake Forest, CA (US); Stanley Kyle Hayes, Mission Viejo, CA (US)

(21) Appl. No.: 12/853,260

(22) Filed: Aug. 9, 2010

Related U.S. Application Data
(60) Provisional application No. 61/232,399, filed on Aug. 7, 2009.

(51) Int. Cl. A61B 17/56

(52) U.S. Cl. 606/86 A

(57) ABSTRACT

Methods, systems, devices and tools for placing bone stabilization components in a patient are provided. The systems and devices have a reduced number of discrete components that allow placement through small incisions and tubes. More particularly, the present invention is directed to systems and methods of treating the spine, which eliminate pain and enable spinal motion. Methods are also provided for stabilizing the thorocolumbar spine region and for implanting the disclosed systems.

### TECHNICAL FIELD

The present disclosure generally relates to surgical instruments and methods for using these instruments. More particularly, but not exclusively, minimally invasive methods of stabilizing one or more bone structures, for example, the thoracic, lumbar and sacral spine, is disclosed.

### BACKGROUND

Systems, methods and devices for stabilizing one or more bone structures of a patient have been available for many years. Securing a metal plate is used to stabilize a broken bone, maintaining the bone in a desired position during the healing process. These implanted plates are either removed when sufficient healing has occurred or left in place for a long-term or indefinite, chronic period. A procedure involving the placement of one or more elongated rods extending between two bone structures or between two components of a single bone structure is often used as a stabilization technique. These rods are placed alongside the bone structure or structures and attached to bone with specialized screws. These procedures require large incisions and also significant tissue manipulation to adequately expose the areas intended for the attachment. The procedures are associated with long recovery times and increased potential for adverse events, such as infection, usually associated with muscle and other tissue trauma and scarring.

Currently available minimally invasive techniques and products are limited, and/or these procedures are difficult to perform, especially in spinal applications in which the attachment points are deeper in tissue, and damage to neighboring tissue must be avoided. Many of the currently available less invasive products remain somewhat invasive due to component configurations, and required manipulations to be performed during the attachment.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings.

FIG. 1A illustrates isometric views of instrumentation used in accordance with aspects of the present disclosure and FIGS. 1B-1L illustrate surgical technique steps for using a bone stabilization system in accordance with aspects of the present disclosure.

FIG. 2 illustrates isometric views of an alignment guide spanner in accordance with aspects of the present disclosure.

FIG. 3 illustrates isometric views of a cap inserter in accordance with aspects of the present disclosure.

FIG. 4 illustrates isometric views of a cap inserter driver in accordance with aspects of the present disclosure.

FIG. 5 illustrates isometric views of an extended compression/distraction tool in accordance with aspects of the present disclosure.

FIG. 6 illustrates isometric views of a rod inserter in accordance with aspects of the present disclosure.

### DETAILED DESCRIPTION

In the following description, certain specific details are set forth in order to provide a thorough understanding of various embodiments of the invention. However, one skilled in the relevant art will recognize that the embodiments of the disclosure may be practiced without one or more of these specific details, or with other methods, components, materials, etc. In other instances, well-known structures associated with bone stabilization devices and systems have not been shown or described in detail to avoid unnecessarily obscuring descriptions of the embodiments of the disclosure. Thus, it is understood that this invention is not limited to particular embodiments described, as such may, of course, vary. It is also understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth. Unless the context requires otherwise, throughout the specification and claims which follow, the word “comprise” and variations thereof, such as, “comprises” and “comprising” are to be construed in an open, inclusive sense, that is as “including, but not limited to.” Furthermore, reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications, patents, patent applications, and other documents mentioned herein are incorporated herein by reference to disclose and describe the methods and/or materials in connection with which the publications are cited. The publications, patents, patent applications and other documents discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present embodiments will now be described in greater detail by way of the following description of exemplary embodiments and variations of the systems and methods of the present invention. While more fully described in the context of the description of the subject methods of implanting the subject systems, it should be initially noted that in certain applications where the natural facet joints are compromised, inferior facet, lamina, posterior arch and spinous process of superior vertebra may be resected for purposes of implantation of certain of the dynamic stabilization systems of the present invention. In other applications, where possible, the natural facet joints, lamina and/or spinous processes are spared and left intact for implantation of other dynamic stabilization systems of the present invention.

It should also be understood that the term “system”, when referring to a system of the present disclosure, most typically refers to a set of components which includes multiple bone stabilization components such as a superior, cephalad or rostral (towards the head) component configured for implantation into a superior vertebra of a vertebral motion segment and an inferior or caudal (towards the feet) component configured for implantation into an inferior vertebra of a vertebral motion segment. A pair of such component sets may include one set of components configured for implantation into and stabilization of the left side of a vertebral segment and another set configured for the implantation into and stabilization of the right side of a vertebral segment. Where multiple bone segments such as spinal segments or units are being treated, the term “system” may refer to two or more pairs of component sets, i.e., two or more left sets and/or two or more right sets of components. Such a multilevel system involves stacking of component sets in which each set includes a superior component, an inferior component, and one or more medial components therebetween.

The superior and inferior components (and any medial components therebetween), when operatively implanted, may be engaged or interface with each other in a manner that enables the treated spinal motion segment to mimic the function and movement of a healthy segment, or may simply fuse the segments such as to eliminate pain and/or promote or enhance healing. The interconnecting or interface means include one or more structures or members that enables, limits and/or otherwise selectively controls spinal or other body motion. The structures may perform such functions by exerting various forces on the system components, and thus on the target vertebrae. The manner of coupling, interfacing, engagement or interconnection between the subject system components may involve compression, distraction, rotation or torsion, or a combination thereof. In certain embodiments, the extent or degree of these forces or motions between the components may be intraoperatively selected and/or adjusted to address the condition being treated, to accommodate the particular spinal anatomy into which the system is implanted, and to achieve the desired therapeutic result.

In certain embodiments, the multiple components, such as superior and inferior spinal components, are mechanically coupled to each other by one or more interconnecting or interfacing means. In other embodiments, components interface in an engaging manner, which does not necessary mechanically couple or fix the components together, but rather constrains their relative movement and enables the treated segment to mimic the function and movement of a healthy segment. Typically, spinal interconnecting means is a dorsally positioned component, i.e., positioned posteriorly of the superior and inferior components, or may be a laterally positioned component, i.e., positioned to the outer side of the posterior and inferior components. The structures may involve one or more struts and/or joints that provide for stabilized spinal motion. The various system embodiments may further include a band, interchangeably referred to as a ligament, which provides a tensioned relationship between the superior and inferior components and helps to maintain the proper relationship between the components.

Reference will now be made in detail to the present embodiments of the disclosure, examples of which are illustrated in the accompanying drawings. Wherever possible, the same reference numbers will be used throughout the drawings to refer to the same or like parts.

The headings provided herein are for convenience only and do not interpret the scope or meaning of the claimed invention.

### A. Overview of Stabilization Device

The following disclosure and figures describe and illustrate several methods and systems for providing an apparatus for stabilizing bone structures. According to aspects of the disclosure, when the system is used as part of a posterior, noncervical pedicle screw system, the stabilization system of the present disclosure provides immobilization and stabilization of spinal segments in skeletally mature patients as an adjunct to fusion in the treatment of the following acute and chronic instabilities of the thoracic, lumbar and sacral spine: degenerative disc disease (DDD) as defined by back pain of discogenic origin with degeneration of the disc confirmed by patient history and radiographic studies; severe spondylolisthesis (Grades 3 and 4) of the L5-S1 vertebrae; degenerative spondylolisthesis; trauma (i.e., fracture or dislocation); spinal stenosis; deformities or curvatures (i.e., scoliosis, kyphosis, and/or lordosis); tumor; pseudoarthrosis; and/or failed previous fusion.

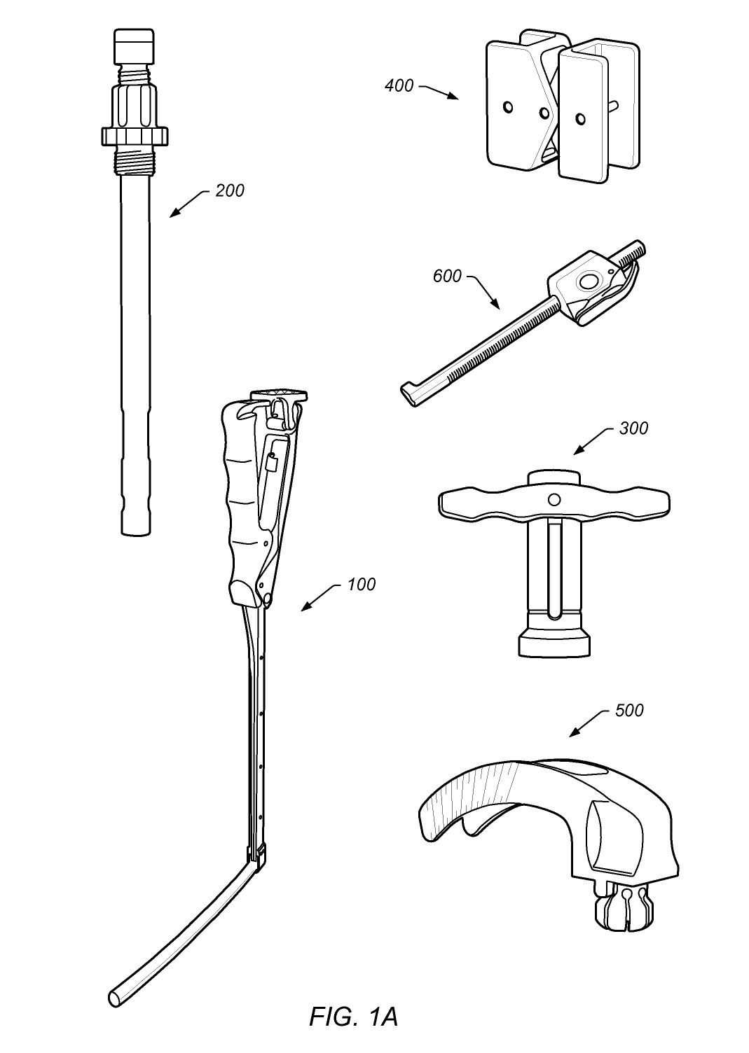

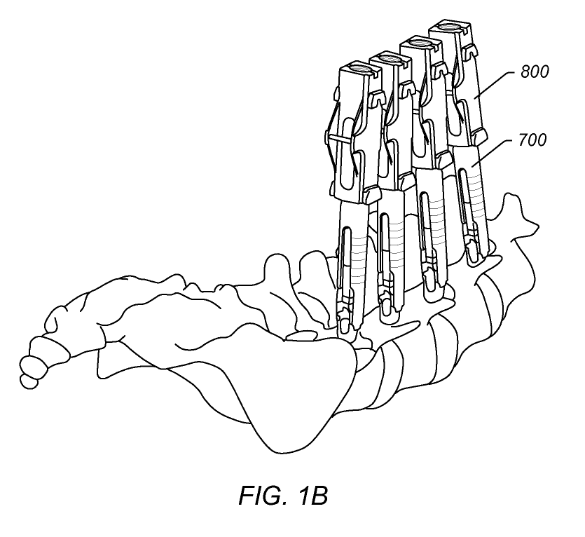

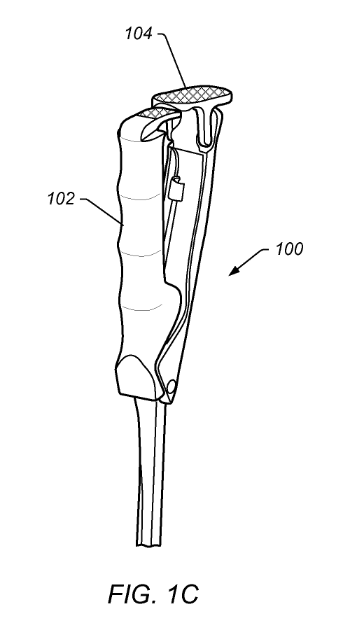

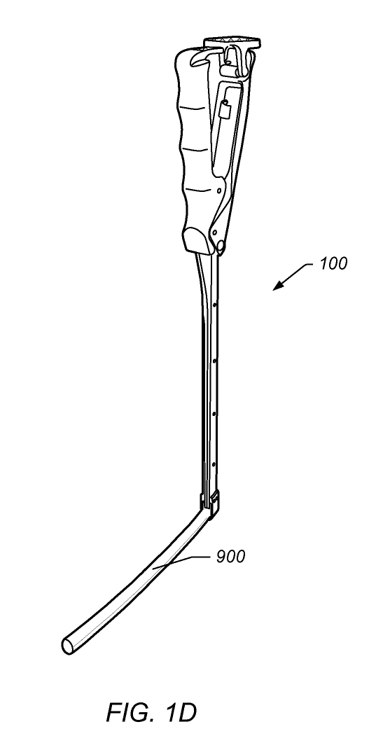

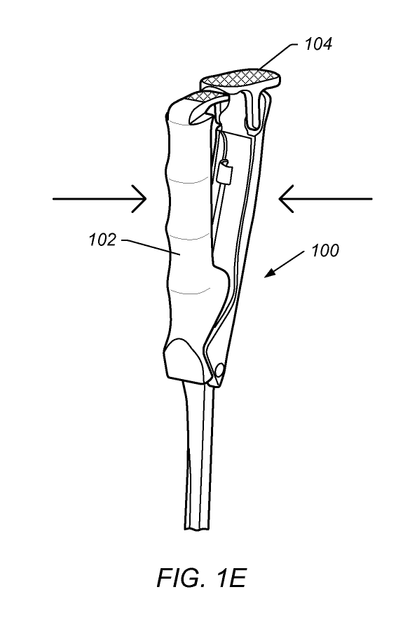

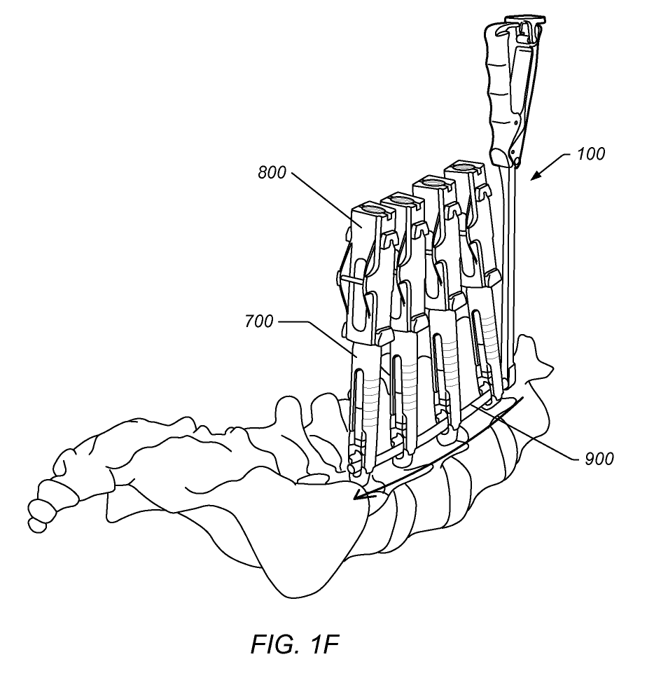

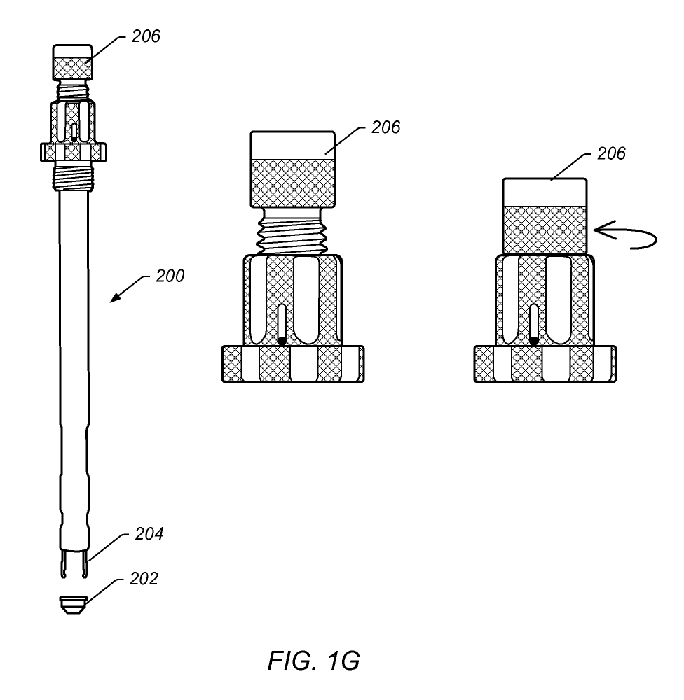

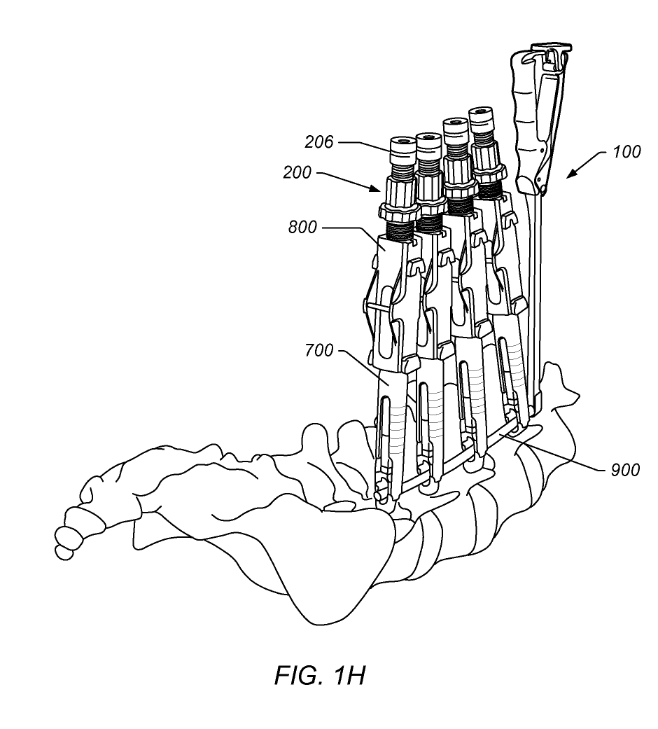

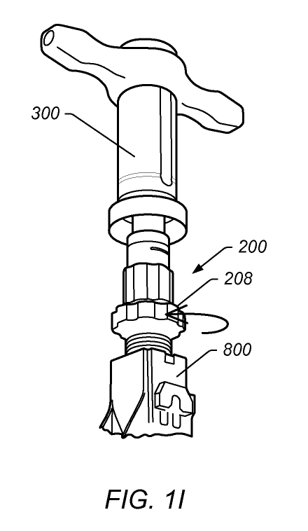

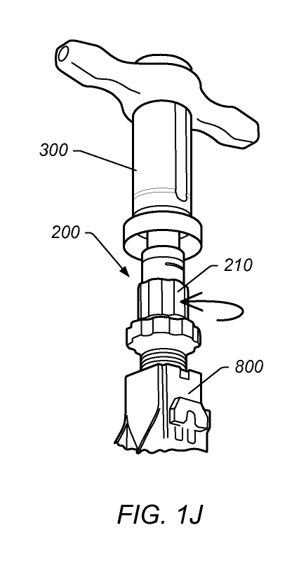

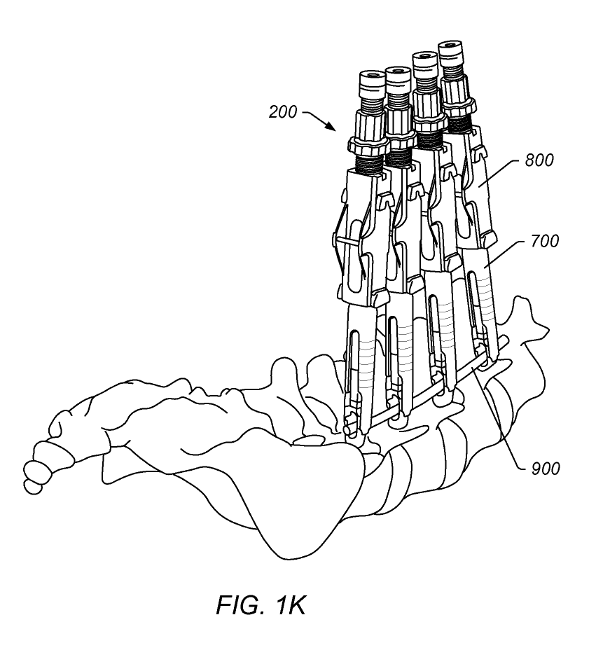

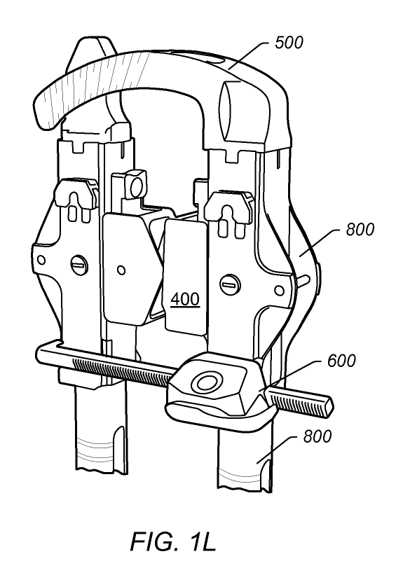

As shown in FIG. 1, the stabilization system described herein includes a rod inserter, a cap inserter, a cap inserter driver, an alignment guide spanner, an extended rod gauge and an extended compression/distraction tool. According to aspects of the disclosure, the stabilization system is designed to enable percutaneous delivery of a standard rod which is longer than 60 mm. Various interchangeable components of the system are described in further detail in co-pending and commonly assigned U.S. patent application Ser. No. 11/586,849, herein incorporated in its entirety by reference. The access assembly and receiving assembly are as generally described in the preceding application; however various improvements and alternative embodiments are disclosed herein, for example, the rod inserter delivers the rod by tunneling through the tissue using the rod inserter while the cap inserters are designed for controlled rod reduction.

According to aspects of the disclosure, and as illustrated in FIGS. 1B-1L, one embodiment of a surgical technique includes: creating an initial minimally invasive incision to accommodate the inserter; targeting the pedicle screw insertion by ensuring the screw depth and medial/lateral placement of screws is as consistent as possible to accommodate the curvature of the rod and minimize contouring. As shown in FIG. 1B, further steps include attaching the towers and alignment guides and then connecting all towers once the alignment guides are attached. As shown in FIG. 1C, further steps include opening the handle of the rod inserter by pushing down on the tab at the top of the inserter and then as shown in FIG. 1D, placing a dynamic rod into the distal end of the rod inserter. If the rod includes a dynamic element, ensure that the dynamic mechanism is closes to the rod inserter. A further step is shown in FIG. 1E including inserting the dynamic rod by gripping the handle of the rod inserter and squeezing to capture and secure the rod into the rod inserter. As shown in FIG. 1F, a further step includes delivering the rod through the superior opening in the cephalad tower and threading the rod through each tower subcutaneously. Once the rod is threaded through each tower, as shown in FIG. 1G, a further step includes assembling the cap to the cap inserter by placing the prongs of the cap inserter over and into the recesses on the cap and turning the top knob clockwise to engage the cap. As shown in FIG. 1H, a further step includes inserting all cap inserters into the alignment guides. As shown in FIG. 1I, a further step includes tightening the cap in the tower furthest from the inserter and turning an actuator to tightening the cap inserter into the alignment guide. If needed, a cap inserter driver may be used. As shown in FIG. 1J, a further step includes locking the cap into the screw. As shown in FIG. 1K, a further step includes sequentially tightening the remaining cap inserters to reduce the rod into place. Finally, as shown in FIG. 1L, a further step includes compressing and/or distracting the vertebra and locking all caps into place.

FIG. 2 illustrates isometric views of an alignment guide spanner in accordance with aspects of the present disclosure. In operation, the alignment guide spanner is attached at a location above the pivot point formed by the primary and secondary alignment guides when it is used to distract the vertebra (for example, by pulling together the rack and pinion) or compress the vertebra (for example, by pushing apart the rack and pinion) Alternatively, the alignment guide spanner is attached at a location below the pivot point formed by the primary and secondary alignment guides when it is used to contract the vertebra (for example, by pulling together the rack and pinion) or distract the vertebra (for example, by pushing apart the rack and pinion). The linear drive mechanism may include an adjustment screw to extend or retract the rack. Rotation of the screw with the screwdriver in one direction causes distraction and rotation in the opposite direction causes compression.

In operation, by extending or retracting the rack in this way a force is applied between the primary and secondary alignment guides. A linear or rotary encoder, or a force measuring transducer, may be provided to increase the precision of the force that is applied and/or the actual measurement of the distraction or compression that is achieved. The force is translated to the screw assemblies, which then impart the force to extend or retract the vertebra to restore disc height to the degenerated or collapsed disc. Once the desired degree of compression or distraction is achieved, the setscrew of the cap assembly is tightened down on the rod to secure the relative position of the screw assemblies. A spring loaded lever serves as a lock and release mechanism on the distraction/compression instrument. The lever engages with the drive mechanism so that it can slide to release the pressure so that the instrument can be removed. In some embodiments of the invention the instrument may also exert a force directly by applying relative torsional forces between them. For instance, the instrument may include its own hinge portion in addition to the linear drive mechanism.

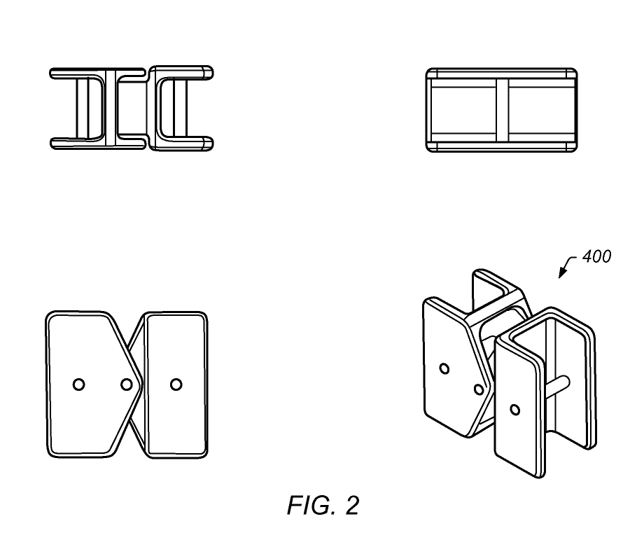

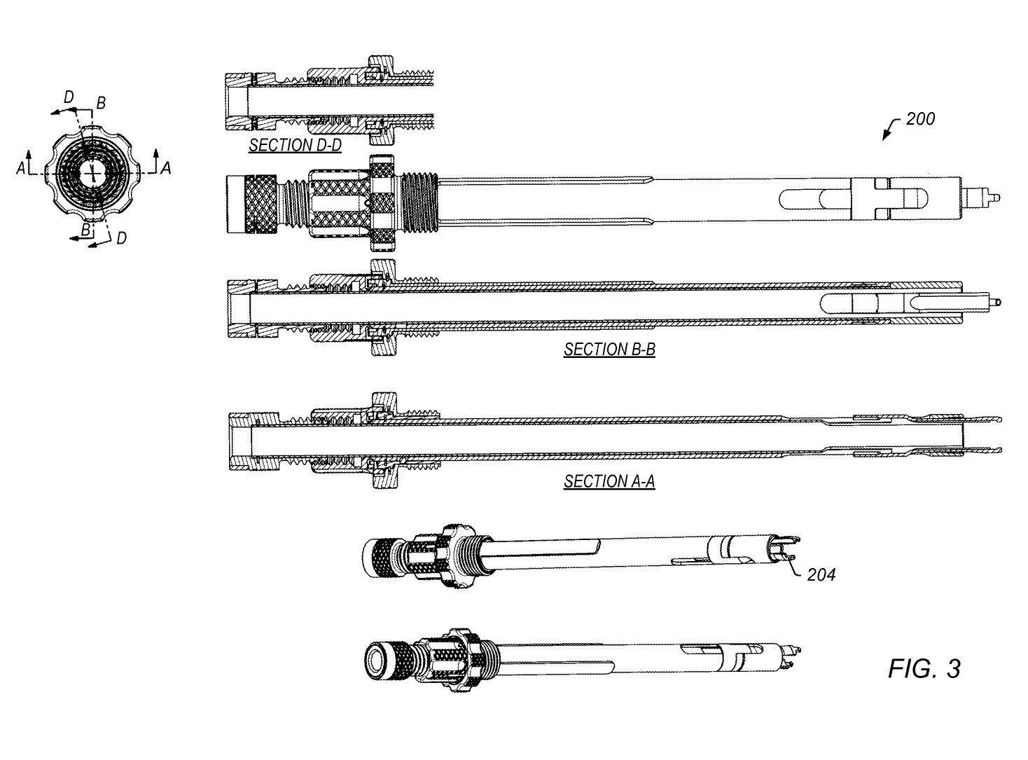

FIG. 3 illustrates isometric views of a cap inserter in accordance with aspects of the present disclosure. FIG. 3 shows a cap inserter instrument that is used to place the cap assembly 300 into the grooves of the seat to secure the end of the rod. As shown, the distal end of the cap inserter has tangs 302 that mate with recesses in the cap assembly to ensure proper orientation so that the cap lugs properly engage with the mating groove in the seat. The tangs may be spring loaded so that they exert a force on the cap assembly to retain it during the insertion. Once the lugs of the cap are in the seat the knob at the proximal end of the instrument is turned to engage the lugs into the grooves of the seat. The knob on the proximal end of the inserter may be knurled for ease in handling and it may also contain a slot for a screwdriver or the like. A threaded collar fits into the top of a secondary alignment guide and is secured in place to ensure that the cap assembly is properly seated for engagement with the seat of the screw assembly. Alternatively, a seating collar with a lug may be used which drops into slots across the top or proximal ends of the primary and secondary alignment guides. The collar also provides mechanical advantage to push the cap before it engages with the screw assembly. The cap assembly 300 is inserted with the setscrew in its remote, fully-retracted position to maximize the room that is available for the rod. The setscrew is dropped into the seat of the screw assembly, where it engages with the grooves prior to being tightened. The knob is rotated (thereby rotating the shaft of the cap inserter instrument 300) until the cap assembly is engaged into the grooves of the seat, which engagement may be indicated to the operator by an audible and/or tactile click.

FIGS. 4A-4G illustrate isometric views of a cap inserter driver in accordance with aspects of the present disclosure. The configuration of the cap inserter driver 400 allows the user to exert additional torque on the cap inserter instrument 300 when for example, the cap assembly does not readily engage with the seat because of tissue that may be in the way.

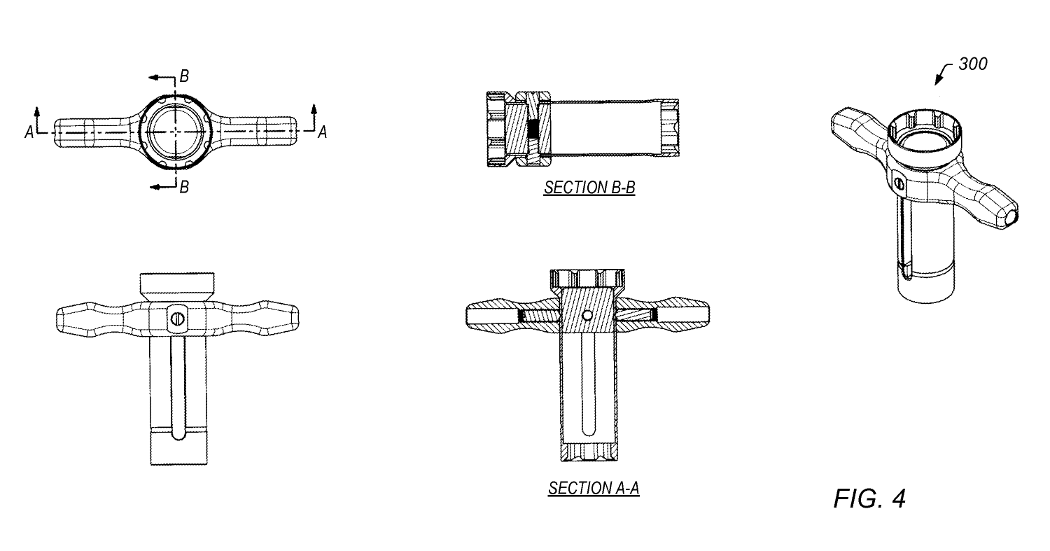

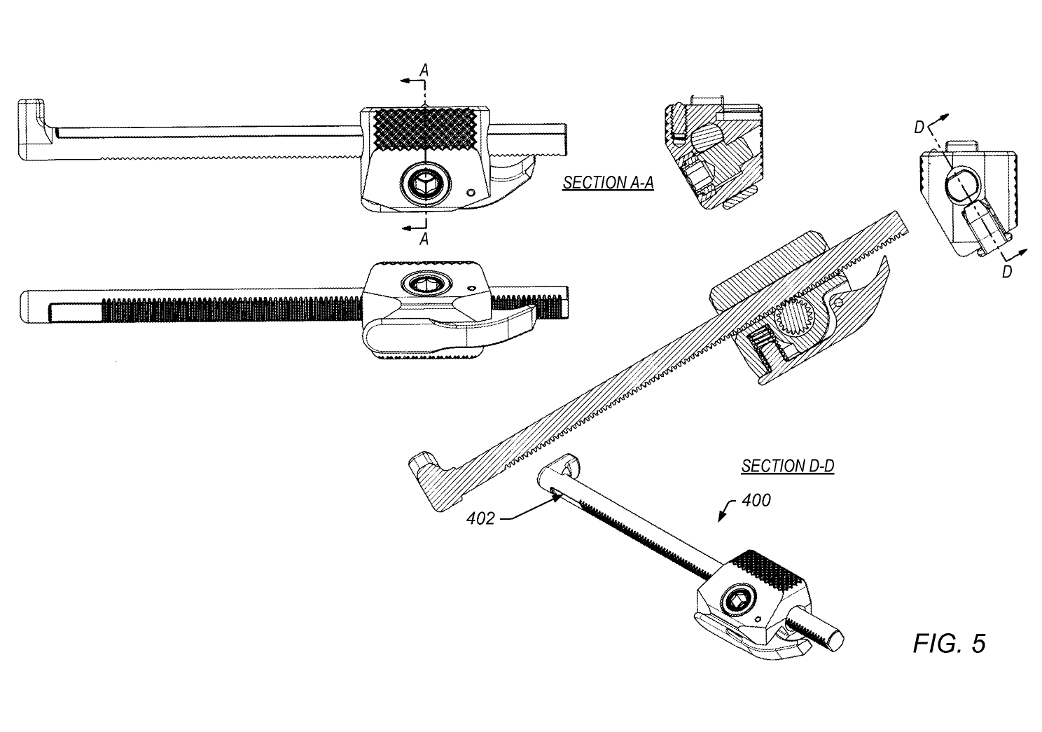

FIG. 5A-5L illustrate isometric views of an extended compression/distraction tool 500 in accordance with aspects of the present disclosure. The distraction/compression instrument 500 may be used to either distract or compress the vertebra to which the bone stabilization device is attached. The distraction/compression instrument 500 attaches to the primary or secondary alignment guides. Specifically, a recess 509 on the back of the distraction/compression instrument 500 slides over and onto a corresponding mating mount on the alignment guides. A ball detent device provides just enough force or resistance to keep the instrument 500 from coming off. In operation, the distraction/compression instrument 500 is fixedly attached to one of the alignment guides. When attached to one of the guides and actuated, the instrument 500 can pull the other guide around the pivot point (i.e., the hook and cross pin) via a lateral post when the rack and pinion are actuated. Alternatively, the instrument 500 can be pivotally attached to both alignment guides, or even integrally formed with either or both of the alignment guides. The instrument 500 includes a rack and pinion or other linear drive mechanism that is translatable along for example, a rack. Of course, other types of drive mechanisms may be employed such as hydraulic/pneumatic or magnetic drives, jack screw drives and rotary gears, for example. The rack then pulls the opposite alignment guide in such a way around the pivot point formed by the hook and cross pin to either distract or compress the vertebra. Depending on whether the distraction/compression instrument 500 is mounted above the pivot point or below the pivot point determines whether distraction or compression is performed

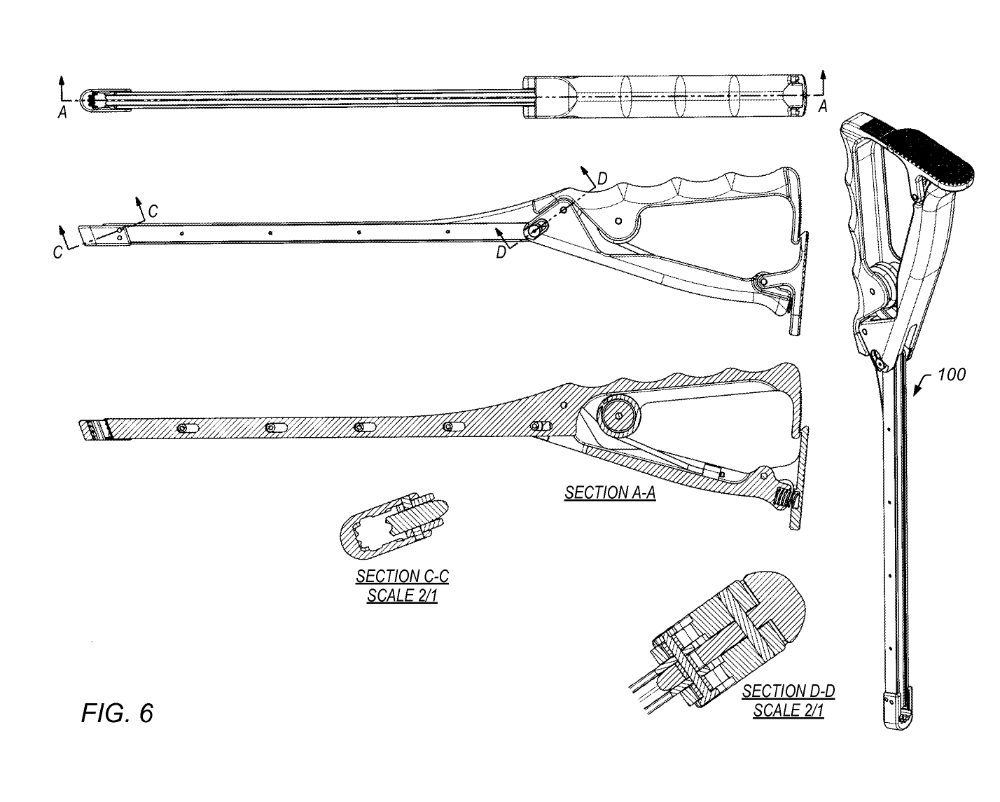

FIG. 6A-6Q illustrate isometric views of a rod inserter assembly in accordance with aspects of the present disclosure. The rod introducer assembly 600 is used to implant the rod after the screw assemblies have been inserted. The rod is slidingly received by the distal end of the assembly and held in place by a frictional fit, possibly with the use of an o-ring that surrounds and compresses the rod. Alternatively, the distal end of the assembly may include threads that engage with the rod to hold it in place. In other cases the distal end of the assembly may be magnetized to hold the rod in place. In yet another alternative embodiment, a separate rod holder may be inserted through the cannula of the rod introducer assembly to hold rod in place. The rod introducer assembly is inserted through the primary or secondary alignment guides and the screw tower assembly and into the coupler of the screw assembly. The proximal end of the introducer assembly includes a rotating collar having external threads received by the threads of the primary and secondary alignment guides. The rotating collar includes notches that mate with the locking tool or other driving and/or pushing tool(s). By rotating the collar the rod is pushed into the coupler. The rod is advanced until it engages with the seat/coupler of the screw assembly. Once the rod is secured the rod introducer assembly is removed. According to aspects of the disclosure, the assembly is configured so that it can only be inserted in a single orientation so that the lugs on the base of the rod properly engages with the coupler and secures the rod to the screw assembly.

According to further aspects of the disclosure, the rod introducer assembly may also be provided with depth, tip and other markings. The markers may include, for example, visible, radiopaque, ultrasonically reflective, or magnetic markers. Other markers or the like may be provided on the shaft of the rod introducer assembly to align it with the primary or secondary alignment guides before it is pushed into the coupler. According to one embodiment, the rod may be pivoted into position so that the rod is engaged with both screw assemblies. The rod pusher fits into the cannula of either the primary or secondary alignment guide. A handle is rotated to pivot the rod toward the second screw assembly. The shaft of the rod pusher is keyed so that it only fits into the cannula with the proper orientation. A threaded collar secures the rod pusher to the secondary alignment guide during the operation. Rotation of the handle turns a pinion to engage and actuate a rack that pushes on a shaft or piston. As the shaft advances it pivots a member on a linkage at the distal tip to drive and pivot the rod into the adjacent screw assembly. This pivoting causes rod to pass through the rod channel in the second alignment guide so that it is received into the coupler of the opposite screw assembly. An indicator in the handle is attached or etched to the rack to show the actuation of the rod pusher. In one embodiment, when the indicator is fully extended toward the proximal end of the handle the rod pusher is fully open. When the indicator is retracted toward the distal end of the handle the rod pusher is fully actuated. Once the rod is in place the rod pusher can be removed by depressing a spring loaded level that unlocks on the rack. Once the release lever is depressed, the rack can be retracted to pull and release the rod pusher. At this point the collar can be disengaged so that the rod pusher can be removed. In some embodiments the rod introducer assembly is included with the rod pusher. In this way the rod introducer assembly does not have to be removed before the rod is pivoted toward the second screw assembly.

In accordance with aspects of the disclosure, the rod pusher may also be provided with depth, tip and other markings. The markers may include, for example, visible, radiopaque, ultrasonically reflective, or magnetic markers. In some embodiments of the invention extensions and/or additional tools may be used to apply an additional mechanical advantage, such as to assist the rod in passing through tissue when the rod is pivoted. For example, a vibrational transducer may be provided which applies micro-pushes or taps to the rod.

Many of the tools described above include one or more engagement means such as matched sets of internal and external threads. Of course, various other types of engagement means may be employed instead, such as press-fits, frictional fits (e.g., tapered fits), bayonet locks and the like. Since a downward force is often applied to the tools (including the engagement means), the tools should be configured to provide a significant mechanical advantage so that a large force can be generated, while allowing the operator to precisely control the force and the distance over which the force is applied. Although it has only been specifically noted with respect to some of the tools described above, any or all of the tools may include markers, which may be visible either with or without equipment. The markers may be used for a variety of purposes, such as to facilitate rotational alignment or orientation (within a single tool, between different tools, and/or between one or more tools and the patient's spine), to measure insertion depth or rod length, to determine engagement or deployment status, or any combination thereof.

The specific details of certain embodiments of the invention are set forth in the following description and in the Figures to provide a thorough understanding of these embodiments to a person of ordinary skill in the art. More specifically, several systems in accordance with embodiments of the invention are initially described with reference to the Figures. A person skilled in the relevant art will understand that the present invention may have additional embodiments, and that the invention can be practiced without several of the details described below.

### B. Applications Incorporated by Reference

The use of methods, components, or elements of the disclosed bone stabilization systems may be employed in combination with various stabilization systems, such as are disclosed in U.S. patent application Ser. No. 11/362,366, entitled Systems and Methods for Stabilization of Bone Structures, herein incorporated by reference in its entirety. Alternatively, the use of methods, components, or elements of the disclosed bone stabilization systems may be employed in combination with still further stabilization systems, such as are disclosed in U.S. patent application Ser. No. 11/726,093, entitled Screw Systems and Methods for Use in Stabilization of Bone Structures; U.S. patent application Ser. No. 11/586,849, entitled Systems and Methods for Stabilization of Bone Structures, and U.S. patent application Ser. No. 12/079,676 entitled Multi-Level Minimally Invasive Spinal Stabilization System, all of which are herein incorporated by reference in their entirety.

### C. Overall Systems and Methods

Bone stabilization systems and devices are operably implanted into a patient. The bone stabilization device may include a hinged assembly which has been attached to first bone segment, and a receiving assembly which has been attached to second bone segment. Bone segments can take on numerous forms, such as two segments from a broken bone such as a femur, tibia and/or fibula of the leg, or the humerus, radius and/or ulna bones of the forearm. In accordance with aspects of the disclosure, bone segments are vertebrae of the patient, such as adjacent vertebra or two vertebra in relative proximity to each other. The device may be implanted to promote healing, reduce or prevent pain, restore motion, provide support and/or perform other functions. The device may be utilized to stabilize bone segments, to prevent or limit movement and/or to dynamically control movement such as to provide restoring or cushioning forces. Accordingly to further aspects of the disclosure, the device is specifically applicable to uses wherein the bone segments are vertebrae of the patient, may stabilize these segments yet dynamically allow translation, rotation and/or bending of these spinal segments, such as to restore an injured or diseased spinal segment to a near-healthy state. According to still further aspects, the device is inserted into a patient, such as a healthy or unhealthy patient, to enhance spinal motion, such as to increase a healthy patient's normal ability to support large amounts of weight, such as for specific military applications, and/or be conditioned to work in unusual environments such as the gravity reduced environments of locations outside earth's atmosphere or at high pressure locations such as in deep-water scuba diving.

According to aspects of the disclosure, the stabilization device may be implanted for a chronic period, such as a period over thirty days and typically an indefinite number of years, a sub-chronic period such as a period greater than twenty-four hours but less than thirty days, or for an acute period less than 24 hours such as when the device is both placed and removed during a single diagnostic or therapeutic procedure. The device may be fully implanted under the skin of the patient, such as when chronically implanted, or may exist both outside the skin and in the patient's body, such as applications where the stabilization components reside above the patient's skin and anchoring screws pass through the skin and attach these stabilization components to the appropriate bone structures.

According to an embodiment of the disclosure, the rod inserter is held at an angle and configured to attach to a rod such that the rod is angled to conform to the lordotic curvature of the spine. According to another embodiment, the rod inserter is substantially parallel to the rod. In operation, the linear or straight configuration of the rod inserter allows the user to visualize the position of the distal portion when in use. According to another embodiment of the disclosure, the tower may be a single unitary design to increase manufacturability and ease of use.

It should be noted that the description and individual drawings herein refers to specific examples of the invention, but that the scope of the invention is not limited except by the eventual scope of any claims directed to the disclosure. Moreover, the sizes and materials shown for the components of the system may vary, but certain ranges of sizes and materials have been shown to be of particular use.

For example, the bone anchors, i.e., pedicle screws, shown may have exemplary lengths ranging from 25 to 80 mm, and may, e.g., be available within that range in 5 mm increments. The diameters of the same may be, e.g., 5.5 mm, 6.0 mm, 6.5 mm, etc. They may be made of metal, such as a titanium alloy, e.g., Ti-6Al-4V, ELI, etc. They may also be made of stainless steel, e.g., 316LSS or 22-13-5SS. The holes into which the same are inserted may be pre-tapped, or alternatively the pedicle screws may be self-tapping. If the bone anchor has a receiving slot, such as a hex head or other such head, then a screwdriver may be used to attach to the bone anchor directly. Once the pivoting rod is in place, a screwdriver may attach to the pivoting rod for further rotation. The pivoting rod itself may be used to further drive the screw. The bone anchors may further have either fixed or polyaxial heads. Their threads may be standard, may be cutting threads, may incorporate flutes at their distal end, or may be any other type of thread. The bone anchors need not be purely of a screw-type. Rather they may also be soft-tissue-type anchors, such as a cylindrical body with a Nitinol barb.

Moreover, the rod, whether dynamic or rigid, may be contoured prior to insertion. In other words, to more closely match the curvature of a spine, or for increased strength, i.e., to accommodate the geometry of the pedicle bone screws, or to accommodate the geometry of the spinal segment in which it is installed, a curve or other contour may be designed into the rod prior to insertion. Alternatively, a physician may bend the rod or put another such contour into the rod, either manually or with the aid of a device, prior to insertion. Furthermore, while the multi-level systems have been shown with rods that are substantially the same size and shape, there is no inherent need for such similarity. The rods can vary in length, diameter, or both. Moreover, the rods can be non-dynamic or can employ dynamic elements.

Further, systems according to the disclosed embodiments may be disposed not only on multiple levels of the vertebrae but also on different sides of the spinous process. In other words, two systems may be disposed in a single segment, one on each pedicle. Moreover, the use of the disclosed pedicle-screw-based systems may be employed in combination with various spacer systems, such as are disclosed in U.S. patent application Ser. No. 11/190,496, entitled Systems and Methods for Posterior Dynamic Stabilization of the Spine, herein incorporated by reference in its entirety.

It should be understood that numerous other configurations of the systems, devices and methods described herein may be employed without departing from the spirit or scope of this application. The pivoting arm of the stabilization device can be attached to bone anchors at its proximal, hinged end, and/or at its translating distal end, with a secured connection that is static (fixed), or it can be secured with a movable, dynamic connection. The pivoting arm and securing connections can be configured to prevent motion of the bone segments, limit motion such as limiting a specific direction or type of motion, or apply specific resistive forces to motion.

The components of the devices of the present invention are preferably configured for percutaneous placement, each device sized for placement through a percutaneous cannula. Each device preferably includes a lumen or sidecar through which a guidewire can be placed or allowing placement along side a percutaneously placed guidewire. The pivoting arm of the present invention can preferably be rotated, such as with the inclusion of a slot allowing the guidewire to exit a lumen, while a guidewire is in place. The pivoting arm and attached components are preferably configured such that the pivoting arm can be secured, such as with insertion of multiple set screws, also with a guidewire in place. Other components may include slot exits from guidewire lumens such as to allow over-the-wire delivery and subsequently escape the guidewire while leaving the guidewire in place. The devices and methods of the present invention are configured to be inserted without resection of tissue, however procedures including or requiring resection are also supported.

The pivoting arm of the present invention preferably includes one or more functional elements. In a preferred embodiment, an artificial facet or facet portion is included and built into the pivoting arm or other component of the bone stabilization device. Each component may include one or more articulating surfaces, such as one located at the end of the pivoting arm and one on either the receiving assembly or hinged assembly of the present invention, such that pre-defined motion between the two attached bone segments can be achieved.

The description of illustrated embodiments and embodiments shown in the figures, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the disclosure can be applied to bone stabilization devices, not necessarily the exemplary bone stabilization devices and systems generally described above.

The various embodiments described and illustrated can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the invention can be modified, if necessary, to employ systems, circuits and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all phase transition liquids and devices that operated in accordance with the claims. Accordingly, the invention is not limited by the disclosure, but instead its scope is to be determined entirely by the following claims.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

1. A system for implanting a spinal stabilization apparatus in a patient, comprising:
a first pedicle access device comprising an elongated tube with a proximal end and a distal end, the proximal end comprising a first engagement means for attachment to a first implantation tool and a second implantation tool;
a second pedicle access device comprising an elongated tube with a proximal end and a distal end, the proximal end comprising a second engagement means for attachment to the first implantation tool;
wherein the spinal stabilization apparatus comprises a first pedicle screw assembly, a second pedicle screw assembly, and a rod configured to be attached between the first pedicle screw assembly and the second pedicle screw assembly; and
at least one of a cap inserter configured for controlled rod reduction and securement.

2. The system of claim 1, wherein the first pedicle access device is configured to be inserted through a minimally invasive procedure.

3. The system of claim 1, wherein the first implantation tool is a rod introducer tool configured to insert the spinal stabilization apparatus rod into the first pedicle screw assembly and the first or second pedicle access device slidingly receives the rod introducer device.

4. The system of claim 3 wherein the rod introducer tool engages the spinal stabilization apparatus rod.

5. The system of claim 3, wherein the rod introducer tool frictionally or magnetically engages the spinal stabilization apparatus rod.

6. The system of claim 3, wherein the first or second pedicle access device slidingly receives the rod introducer tool through a mechanical key configured to rotationally orient the rod introducer tool.

7. The system of claim 3, wherein the rod introducer tool is configured to deliver a rod longer than 60 mm.

8. The system of claim 1, further comprising a third pedicle screw assembly.

9. The system of claim 1, further comprising a third pedicle screw assembly, wherein the first, second and third pedicle screw assemblies are attached with an alignment guide spanner.

10. The system of wherein the first implantation tool is a rod introducer tool configured to insert the spinal stabilization apparatus rod into the first pedicle screw assembly and the first or second pedicle access device slidingly receives the rod introducer device, and wherein the rod inserter includes a handle having a release mechanism, the release mechanism having a first position and a second released position, wherein the rod is retained when the release mechanism is in a first position and the rod slideably releases when the release mechanism is in the second position.

11. The system of claim 1, wherein the cap inserter includes prongs at a distal end and an engagement mechanism at a proximal end, wherein the engagement mechanism has a first engaged position and a second released position such that the prongs are locked onto the associated pedicle screw assembly when the engagement mechanism is in a first engaged position.

12. The system of claim 11, wherein the prongs are retractable.

13. The system of claim 11, wherein each pedicle screw assembly has an associated cap inserter.

14. The system of claim 1, wherein the cap inserter further comprises a first adjustment device and a second adjustment device, wherein the first and the second adjustment devices are independently adjustable.

15. The system of claim 14, wherein the first adjustment device has an engaged position and a disengaged position.

16. The system of claim 14, wherein the second adjustment device is rotationally engaged.

17. A method of treating the thorocolumber spine region, comprising:
implanting a first bone anchor assembly in a first vertebrae;
implanting a second bone anchor assembly in second vertebrae;
providing a first access device comprising an elongate tube with a proximal end and a distal end;
inserting the first access device through a first incision;
connecting the distal end of the first access device to the first bone anchor assembly;
providing a second access device comprising an elongated tube with a proximal end and a distal end;
inserting the second access device through a second incision;
connecting the distal end of the second access device to the second bone anchor assembly;
aligning the first bone anchor assembly and the second bone anchor assembly;
inserting a connecting rod into the first access device at the proximal end;
tunneling through tissue by moving the connecting rod inside the first access device from the proximal end to the distal end of the first access device;
aligning the connecting rod with the first and second bone anchor assemblies;
engaging cap inserters configured for controlled rod reduction to connect the connecting rod to the first and second bone anchor assemblies;
disconnecting the first and second access devices from the first and second bone anchor assemblies; and
removing the first and second access devices.

18. The method of claim 17, wherein the first and second incision are the same incision.

19. The method of claim 17, wherein the cap inserters are sequentially engaged to reduce the rod into place.

20. The method of claim 17, further comprising locking the cap inserters to the bone anchor assemblies with a locking device and then sequentially tightening the cap inserters with an adjustment device to reduce the rod into place.
